(19) United States
(12) Patent Application Publication
(10) Pub. No.: US 2022/0008490 A1
(43) Pub. Date: Jan. 13, 2022

(54) COMPOSITION FOR THE PREVENTION AND TREATMENT OF SKIN DISEASES

(71) Applicant: DRN S.R.L., Palazzo Pignano (CR) (IT)

(72) Inventors: Gennaro Falanga, Palazzo Pignano (CR) (IT); Patrizio Conselvan, Palazzo Pignano (CR) (IT); Elisa Cavazzuti, Palazzo Pignano (CR) (IT)

(21) Appl. No.: 17/413,306

(22) PCT Filed: Dec. 12, 2019
(86) PCT No.: PCT/IB2019/060700

§ 371 Date: Jun. 11, 2021

(30) Foreign Application Priority Data
Dec. 13, 2018 (IT) 102018000011056

(51) Int. Cl. A61K 35/747; A61K 36/48; A61K 36/899; A61K 36/03; A61K 36/05; A61K 9/10; A61K 9/00

(52) U.S. Cl. CPC A61K 35/747; A61K 36/48; A61K 36/899; A61K 2035/115; A61K 36/05; A61K 9/10; A61K 9/0014; A61K 36/03

(57) ABSTRACT

A composition is described for the prevention and treatment of skin diseases, in particular in the veterinary field. The composition of the invention includes a synergistic association of Lactobacilli and plant extracts, which has been shown to be significantly effective in supporting the functionality of the skin barrier and restoring homeostasis of microbial populations. In fact, the composition performs a direct action on the skin surface by preventing and contrasting skin alterations, promoting the balance of the microbiota, strengthening the immune system and carrying out antioxidant activity.

### FIELD OF THE INVENTION

The present invention relates to a composition for the prevention and treatment of skin diseases, in particular in the veterinary field. The composition of the invention comprises a synergistic association of Lactobacilli and plant extracts, which has been shown to be significantly effective in supporting the functionality of the skin barrier and restoring homeostasis of microbial populations. In fact, the composition performs a direct action on the skin surface by preventing and contrasting skin alterations, promoting the balance of the microbiota, strengthening the immune system and carrying out antioxidant activity.

### BACKGROUND ART

In recent years, the incidence of allergies in urbanized countries and among the population with “western” lifestyle has been progressively increasing. There are many evidences that underline how, in humans, a varied bacterial exposure, especially in the first years of life, for example through contact with animals or other family members, contributes to the development of a healthy microbiota, reducing the onset of allergies.

The mechanisms underlying these three factors, namely the environment, allergies and microbiota, however, remain to be fully determined.

The studies conducted so far in this sector have been based on humans or on mouse models. In the first case, there are too many possible confounding factors to consider in interpreting the results (lifestyle changes from person to person or during the phases of life, travel etc.), in the second case, instead, there are models too simplified and “artificial”.

With the aim to deepen the topic with evidence more transferable to the human context, some researchers have opted for dog models as they are much more integrated into our daily lives than murine ones, often going to share the same lifestyle and environment of their owners, but with minor variables.

To assess the impact of the environment on the bacterial skin component, three distinct factors were considered, namely the context of residence, movement and lifestyle. All three aspects considered were related to the skin microbiota.

Allergies were more common than double in dogs living in an urban context, adopting their lifestyle compared to those of rural areas and lifestyles.

The skin microbiota of dogs belonging to healthy dog families differs from that of dogs with allergic relatives.

In conclusion, therefore, the skin microbiota of healthy dogs is different from that of allergic dogs. The environment and lifestyle, even in the early years of life, are fundamental in determining the characteristics of the skin microbiota in dogs and, consequently, also their risk of developing allergies or not. Further studies will however be necessary in order to determine the cause-effect relationship, however, to date, allergies, inflammatory states and skin changes, such as atopic dermatitis, are typically treated with antibiotics.

It is therefore an object of the present invention to provide a solution to support conventional pharmacological therapies in order to achieve a more rational use of the use of antibiotics, which allows to prevent and effectively treat skin diseases, without triggering resistance phenomena typical of antibiotics, and being at the same time of practical use.

### SUMMARY OF THE INVENTION

Said object has been achieved by a composition comprising Lactobacilli and plant extracts, as reported in claim 1.

In another aspect, the present invention relates to the use of this composition for the prevention and treatment of skin diseases, in particular in the veterinary field.

In a further aspect, the present invention relates to the use of Lactobacilli for the prevention and treatment of skin diseases, particularly in the veterinary field.

With the term “skin diseases”, it is meant to include spots, papules, blisters, blebs, pustules, cysts, erosions, abrasions, redness, ulcers, cracks, sores, telangiectasia, flaking, erythema, crusts, lichenifications, grazes, hardenings, cuts, lacerations, or atrophies. These skin diseases are the manifestation of skin diseases such as dermatitis, atopic dermatitis, digital and interdigital dermatitis, allergy, pododermatitis, pyoderma, dermatosis, furunculosis, candidiasis, erythroderma, erythema, burns, folliculitis, trichomycosis, keratitis, eczema, psoriasis, porokeratosis, urticaria, demodectic mange, malasseziasis, parasitosis.

### BRIEF DESCRIPTION OF THE DRAWINGS

The characteristics and advantages of the present invention will become clear from the following detailed description, from the embodiments provided by way of illustrative and non-limiting examples, and from the attached figures, wherein:

FIG. 1 illustrates a first result of the comparative test carried out in Example 3,

FIG. 2 illustrates other results of the comparative test carried out in Example 3, and

FIG. 3 illustrates the comparison between the composition of the invention and the comparative composition both before (A) and after (B) centrifugation, as per Example 3.

### DETAILED DESCRIPTION OF THE INVENTION

The invention therefore relates to a composition comprising:
at least one Lactobacillus selected from Lactobacillus rhamnosus, Lactobacillus reuteri and their mixtures,
at least one plant extract selected from tamarind extract, sugar cane extract and their mixtures,
   wherein said at least one plant extract and said at least one Lactobacillus are in a weight ratio of 1.1:1 to 10:1.

It has indeed been surprisingly found that the association of at least one Lactobacillus and at least one plant extract allows to support the functionality of the skin barrier and the restoration of homeostasis of microbial populations. In fact, the composition performs a direct action on the skin surface by preventing and contrasting skin alterations, promoting the balance of the microbiota, strengthening the immune system and carrying out antioxidant activity.

The association of the components of the invention allows to exploit both the synergistic action that they exert among themselves, and the amplification of the functions of the individual components due to the interaction with the substrate on which they are applied (e.g. keratinocytes, microbiota, immune system). This composition therefore fits well within a modern method of multimodal approach to the treatment of chronic diseases, especially on an allergic basis, and has the aim of allowing management of this type of problem by integrating and saving, as far as possible, the use of drugs that could have side effects, as well as the increasing problem of antibiotic resistance or the problems related to the chronic use of corticosteroid drugs and immunomodulatory drugs.

The synergy of action among the components allows to increase effectiveness by acting on several fronts simultaneously, namely:
selection of beneficial microbial skin populations,
protection from oxidative stress both at the level of skin cells and microbial populations,
application of prebiotics that select beneficial microbial populations,
induction to the production of substances that improve the integrity of the skin barrier (by interaction with beneficial microbial populations).

In particular, said at least one Lactobacillus acts at the modulation level of skin microbial populations following topical application in the presence of skin dysbiosis, which occurs, for example, during allergic dermatitis in dogs and cats. This modulation activity takes place in different ways, some of which being not yet fully clarified. The topical application of Lactobacillus rhamnosus and Lactobacillus reuteri is able to perform a competitive inhibition activity against Staphylococcus pseudointermedius, but also to develop an interaction with microbial populations and the cutaneous immune system. The result is a modulation of the cutaneous microbial populations which leads to the development of a cutaneous microenvironment unfavorable to the proliferation of S. pseudointermedius.

The modulation of skin microbial populations can lead to a decrease in the symptoms associated with allergic dermatitis because, by creating an unfavorable environment for the development of S. pseudointermedius, it reduces for example the itching associated with both the presence of the bacterium and the activity of its metabolites and/or toxins. The proliferation of beneficial microbial skin populations that occurs following the topical application of Lactobacillus rhamnosus and Lactobacillus reuteri allows to reduce the severity of the clinical symptomatology in the course of allergic dermatitis also through the interaction of these beneficial skin populations with the immune system and through the production of inflammation mediators and skin antimicrobial peptides.

The beneficial bacterial populations of the skin constitute a fundamental component of the skin barrier and, through the modulation activity of these populations, the composition of the invention allows to improve the symptomatology in course of allergic dermatitis of the dog and cat. Lactobacillus rhamnosus and Lactobacillus reuteri act in synergy both with the other components of the composition, and with the concomitant therapies (pharmacological and non-pharmacological, topical and systemic) that are used classically in the course of allergic dermatitis in dogs and cats.

The polyphenols present in the sugar cane extract exhibit an antioxidant activity of fundamental importance in the course of allergic dermatitis and all those skin conditions associated with the production of free radicals (inflammation, infection, autoimmune diseases). Polyphenols are able to inhibit the growth of some pathogenic and/or potentially pathogenic bacterial populations (including S. aureus) and to promote the proliferation of beneficial bacteria, including Lactobacilli. The polyphenols have also been shown to protect some beneficial microbial populations through surface interactions that make them less subject to oxidative stress during inflammatory and infectious processes. This feature allows to increase the stability of the composition of the invention, providing adequate protection to Lactobacillus rhamnosus and Lactobacillus reuteri. The tamarind extract allows to increase skin hydration in depth by promoting the reduction of TEWL (i.e. transepidermal water loss) which occurs during allergic dermatitis. The increase in skin hydration has a positive effect on the microbial populations adhering to the surface of the keratinocytes. The xyloglucans contained in the extract also act as a vehicle and promote the penetration of polyphenols deep into the skin layers, thus increasing their antioxidant efficacy.

Finally, as demonstrated in the following Examples, it has been observed that the association of said at least one plant extract and said at least one Lactobacillus in the indicated weight ratios offers a significant stability over time not only in static conditions, but also even when subjected to external extreme stresses, such as centrifugation. Preferably, said at least one plant extract and said at least one Lactobacillus are in a weight ratio of 1.2:1 to 5:1.

Preferably, the composition of the invention comprises up to 5 wt % of said at least one Lactobacillus.

For the purposes of the present invention, “wt %” means % by weight based on the weight of the composition of the invention.

More preferably, the composition of the invention comprises 0.1-3 wt % of said at least one Lactobacillus.

Preferably, said at least one Lactobacillus is in a concentration of 10-200 billion CFU/g. Preferably, the composition of the invention comprises up to 10 wt % of said at least one plant extract.

More preferably, the composition of the invention comprises 0.2-6 wt % of said at least one plant extract.

In preferred embodiments, the composition of the invention comprises a mixture of Lactobacillus rhamnosus and Lactobacillus reuteri. Preferably, Lactobacillus rhamnosus is in greater quantities than Lactobacillus reuteri. More preferably, Lactobacillus rhamnosus and Lactobacillus reuteri are in a weight ratio of 4:1 to 1.2:1.

Preferably, the composition of the invention comprises up to 3 wt % of said Lactobacillus rhamnosus, more preferably 0.2-2 wt %.

Preferably, the composition of the invention comprises up to 2 wt % of said Lactobacillus reuteri, more preferably 0.1-1.5 wt %.

In some embodiments, said at least one Lactobacillus is freeze-dried. The freeze-drying process allows the vitality of the bacteria to be maintained, which reactivate following rehydration which takes place once in contact with the skin surface.

In particularly preferred embodiments, said at least one Lactobacillus is tyndallized. With the term “tyndallized”, it is meant subjected to a particular heat treatment that has inactivated it, therefore rendered it incapable of metabolizing and reproducing itself. Lactobacilli are treated together with their culture medium, constantly checked and optimized, which also contains the substances produced by their metabolism. The lyophilisate thus obtained therefore contains both the probiotic cellular parts and the substances of the normal metabolism of the strains (vitamins, glycoproteins, micronutrients), therefore it can be as effective as the contribution of the same live strains. As mentioned, the activity of a tyndallized Lactobacillus cannot be ascribed to the possibility of generating new offspring and directly colonizing a physiological site. Differently, its contribution constitutes a basis on which a substrate suitable for the recolonization of the symbiotic flora is reconstituted and at the same time there is a contrast to the establishment and development of all pathogenic or harmful strains. This seems to be due to the macromolecules that make up the bacterial cell walls of the tyndallized (Bacteriocins) and to those coming from their cytoplasm (DNA, enzymatic proteins, vitamins, etc.) which carry out a supporting and stimulating action towards the proliferation of beneficial bacteria while counteracting the development of pathogenic ones.

The use of tyndallized Lactobacilli allows to obtain:
direct bacteriostatic activity, by preventing or delaying the development of pathogenic bacteria,
support for the development of the acidogenic defense, mainly due to the presence of B vitamins,
adhesion of the tyndallized cell fragments to the epithelial cells with an inhibiting effect on the adhesiveness and expansion of pathogenic strains, and
stimulation of the immune system.

As for the plant extract, in preferred embodiments, the composition of the invention comprises a mixture of tamarind extract and sugar cane extract. Preferably, the tamarind extract is in a greater quantity than the sugar cane extract. More preferably, tamarind extract and sugar cane extract are in a weight ratio of 15:1 to 5:1.

Preferably, the composition of the invention comprises up to 8 wt % of said tamarind extract, more preferably 0.5-6 wt %.

Preferably, the composition of the invention comprises up to 2 wt % of said sugar cane extract, more preferably 0.1-1 wt %.

Said tamarind extract is preferably a glycolic tamarind extract. In preferred embodiments, the extraction solvent is a mixture of propylene glycol and water.

Tamarind belongs to the legume family and contains a natural branched polysaccharide (Xyloglucan) with the ability to form viscous and moisturizing gels. Xyloglucans, molecules with a weight of 650,000 Daltons, bind water, retain it and release it into the skin, thus obtaining a moisturizing effect superior to that of hyaluronic acid. In particular, they exert an effect on the activity of filaggrin, a protein important for maintaining skin hydration. This protein catalyzes the formation of disulfide bridges between the keratin filaments in the stratum corneum inducing a greater retention of water which results in greater hydration. Furthermore, Tamarind has a high content of polyphenols with a high antioxidant activity.

Said sugar cane extract is preferably a polyphenolic extract in which at least 50% of polyphenols has an average numerical molecular weight of 600-2000 Daltons, as measured by size exclusion chromatography. In preferred embodiments, the extraction solvent of said sugar cane extract is propylene glycol.

The polyphenols contained in this extract are powerful antioxidants, protect cells from damage caused by UV rays and contribute to the modulation of the skin microenvironment by counteracting inflammatory processes.

Preferably, the composition of the invention further comprises at least one algae extract. The latter carries out a selective prebiotic activity for beneficial skin microbial populations, and also provides essential micronutrients for the well-being of the skin. The algae extract inserted in the context of the composition of the invention contributes effectively to the biodiversity of skin microbial populations, by promoting skin replication and probiotic activity, in synergy with Lactobacillus rhamnosus and Lactobacillus reuteri.

Preferably, the composition of the invention comprises up to 2 wt % of said at least one algae extract.

More preferably, the composition of the invention comprises 0.1-1 wt % of said at least one algae extract.

More preferably, said algae extract is an extract of Laminaria digitata, an extract of Chlorella vulgaris or a mixture thereof.

Laminaria digitata is a brown alga rich in iodine and sodium, dark brown in color and large in size. It is found in cold seas and oceans, particularly in the North Sea and the North Atlantic. It is found in abundance along the coasts of Great Britain and Ireland, Iceland and Russia and Brittany.

Laminaria digitata extract is preferably obtained by leaching with water, filtration and concentration by reverse osmosis.

Chlorella vulgaris is a blue-green alga that grows mainly in the brackish waters of Equatorial Africa. Its particular green color is due to the presence of a significant concentration of chlorophyll. Among the most important nutrients present in this alga are vitamins, especially of the groups A, B, C and E, as well as minerals such as iron, calcium, magnesium and phosphorus. The alga also contains carotenoid substances capable of counteracting the harmful action of free radicals. Rich in fiber, it has a high content of high quantity proteins. Among the amino acids present, there are in particular leucine, tyrosine, lysine and phenylalanine.

The Chlorella vulgaris extract is preferably obtained by enzymatic extraction in water and subsequent thermal inactivation, filtration and neutralization of the pH.

In preferred embodiments, the composition of the invention comprises a mixture of Laminaria digitata extract and Chlorella vulgaris extract.

The mixture of brown and green algae brings a variety of nutrients that allow to ensure the biodiversity and homeostasis of the microbiota of the skin, namely:
sugars and sugar alcohols for energy,
peptides and amino acids as nitrogen sources for bacterial replication,
exopolysaccharides useful for their implication in intracellular communication, and
mineral salts for the development of a differentiated microflora.

These aspects constitute evidence of the synergy between the algae extracts that perform prebiotic activity and the lactic acid bacteria, in particular tyndallized, which stimulate the response of skin bacterial populations, in terms of biodiversity.

Preferably, the extract of Laminaria digitata is in a greater quantity than the extract of Chlorella vulgaris. More preferably, the extract of Laminaria digitata and the extract of Chlorella vulgaris are in a weight ratio of 5:1 to 2:1.

In some embodiments, the composition of the invention further comprises vitamin E. Preferably, the composition comprises up to 2 wt % of vitamin E.

The composition of the invention can further comprise pharmaceutically acceptable excipients. The term “excipient” means a compound or a mixture thereof suitable for use in a composition for external topical use. For example, an excipient generally should not cause an adverse response in a subject, nor should it significantly inhibit the effectiveness of the composition itself.

Suitable excipients can be rheological additives, pH regulators, antioxidant agents, anti-seborrheic agents, antistatic agents, absorbent agents, UV absorbing agents, astringent agents, skin conditioning agents, preserving agents, covering agents, denaturing agents, depigmenting agents, emulsifying agents, agents film-forming agents, gelling agents, moisturizing agents, hydrotropic agents, soothing agents, smoothing agents, matting agents, plasticizing agents, propellants, skin protective agents, reducing agents, refreshing agents, sebum-restoring agents, solvents, emulsion stabilizing agents, toning agents, agents wetting agents, and their mixtures.

Among solvents, water is particularly preferred, as well as oils.

The addition of excipients can be carried out by methods known in the art. In fact, the components can, for example, be mixed as such or with one or more excipients. The composition of the invention can be in the form of a solution, emulsion, suspension, gel, or spray.

Preferably, the composition is in the form of a spray, wherein the solvent is water.

Particularly preferred are those compositions in the form of aqueous sprays, comprising:
0.2-2 wt % of tyndallized Lactobacillus rhamnosus
0.1-1.5 wt % of tyndallized Lactobacillus reuteri
0.5-6 wt % of sugar cane extract
0.1-1 wt % of tamarind extract
0.1-1 wt % of a mixture of Laminaria digitata extract and Chlorella vulgaris extract.

Preferably, the composition is in the form of a suspension, wherein the solvent is an oil. Vegetable oils are preferred, in particular, coconut oil, palm oil, olive oil, safflower oil, sunflower oil, soybean oil, soybean oil, flaxseed oil, oilseed rape, evening primrose oil, and mixtures thereof. Particularly preferred are coconut oil, palm oil, and mixtures thereof.

Particularly preferred are those compositions in the form of an oil-based suspension, comprising:
0.2-2 wt % of Lactobacillus rhamnosus
0.1-1.5 wt % of Lactobacillus reuteri
0.5-6 wt % of sugar cane extract
0.1-1 wt % of tamarind extract
up to 2 wt % of vitamin E.

In some embodiments, the composition of the invention essentially consists of at least one Lactobacillus, at least one plant extract, and optionally at least one algae extract, as described above. The expression “essentially consists of” means that these are the only active ingredients for the prevention and treatment of skin diseases to be present in the composition of the invention, while any further components or excipients do not interfere with these active ingredients.

In other embodiments, the composition of the invention consists of at least one Lactobacillus, at least one plant extract, and optionally at least one algae or vitamin E extract, and pharmaceutically acceptable excipients, as described above.

It should be understood that all the aspects identified above as preferred and advantageous for the composition and its components, are to be considered analogously preferred and advantageous also for these embodiments.

In another aspect, the present invention relates to the use of the composition described above for the prevention and treatment of skin diseases.

In fact, as mentioned, the association of at least one Lactobacillus and at least one plant extract allows to support the functionality of the skin barrier and the restoration of homeostasis of microbial populations. In fact, the composition performs a direct action on the skin surface by preventing and contrasting skin alterations, by promoting the balance of the microbiota, strengthening the immune system and carrying out antioxidant activity.

In a further aspect, the present invention relates to the use of tyndallized Lactobacillus for the prevention and treatment of skin diseases, in particular in the veterinary field. The composition of the invention is preferably administered topically externally. Preferably, said skin conditions are in animal beings, such as pets. In particular, these pets are dogs and cats.

It should be understood that all the possible combinations of the preferred aspects of the components of the composition, as indicated above, are deemed to be hereby described, and therefore similarly preferred.

It should be also understood that all the aspects identified as preferred and advantageous for the composition and its components are to be considered analogously preferred and advantageous also for the preparation and the uses of the composition itself.

Working examples of the present invention are given below for illustrative and non-limiting purposes.

### EXAMPLES

### Example 1

The following solution has been prepared, according to the present invention:

| | wt % |
|---|---|
| tyndallized Lactobacillus | 1 (150 billion UFC/g; 1.5 billion UFC/ml) |
| rhamnosus | |
| tyndallized Lactobacillus | 0.5 (100 billion UFC/g; 500 million UFC/ml) |
| reuteri | |
| Sugar cane extract | 0.25 |
| (from propylene glycol) | |
| Tamarind extract | 2 |
| (from propylene glycol | |
| and water) | |
| Actibiome PE(*) | 1 |
| (by Codif, France) | |
| excipients | 1.2 |
| water | balance to 100.00 |

(*) comprises 1.25% of Laminaria digitata extract and 0.39% of Chlorella vulgaris extract

The solution of the present Example can be administered in a spray form directly on the skin to be treated, preferably 1-2 times a day.

### Example 2

The following oily suspension has been prepared, according to the present invention:

| | wt % |
|---|---|
| Lactobacillus | 0.5 (150 billion UFC/g; 750 million UFC/ml) |
| rhamnosus SGL06 | |
| (freeze-dried) | |
| Lactobacillus | 0.25 (>100 billion UFC/g; 250 million UFC/ml) |
| reuteri SGL01 | |
| (freeze-dried) | |
| Sugar cane extract | 0.25 |
| (from propylene | |
| glycol) | |
| Vitamin E | 0.1 |
| Tamarind extract | 2 |
| (from propylene | |
| glycol and water) | |
| Cetiol ® CC | 50 |
| Cetiol ® OE | 46.90 |

An oily suspension was obtained for topical application, containing freeze-dried Lactobacilli and vegetable polyphenols, with modulation activity of skin microbial populations.

The oily suspension of the present Example can be administered directly on the skin to be treated, preferably 1-2 times a day.

The presence of the specific oils included in the formulation allows to preserve the characteristics of the two populations of Lactobacilli, thus preventing their replication within the product and thus ensuring its stability. In other words, the freeze-drying process acts in synergy with the oils in order to obtain a stability that allows the reactivation of the bacterial strains only at the application site at the skin level.

Cetiol® CC (supplied by BASF, comprising dicaprilyl carbonate from palm oil and coconut oil) and Cetiol® OE (supplied by BASF, comprising dicaprilyl ether from palm oil and coconut oil) are two oils able to guarantee rapid absorption following topical application and they convey the components of the product both on the surface and in depth through the skin layers. In addition, both oils have the characteristic of ensuring a large diffusion area on the skin surface. The latter aspect is considered particularly important when it comes to the topical application of products in pets (in particular dogs and cats), as it allows to guarantee the distribution of the active ingredients at a distance from the point of application even in the presence of piliferous coverage.

### Example 3

The following comparative composition has been prepared:

| | wt % |
|---|---|
| tyndallized | 1 (150 billion UFC/g; 1.5 billion UFC/ml) |
| Lactobacillus | |
| rhamnosus | |
| tyndallized | 0.5 (100 billion UFC/g; 500 million UFC/ml) |
| Lactobacillus | |
| reuteri | |
| Sugar cane extract | 5 |
| (from propylene | |
| glycol) | |
| Tamarind extract | 14.5 |
| (from propylene | |
| glycol and water) | |
| Actibiome PE(*) | 1 |
| (by Codif, France) | |
| excipients | 1.2 |
| water | balance to 100.00 |

The composition of Example 1, briefly “[INV]”, and the comparative composition, briefly “[comp.]” differ only in the weight ratio between plant extracts and Lactobacilli. In fact, in the case of the composition [comp.], this ratio is 13:1.

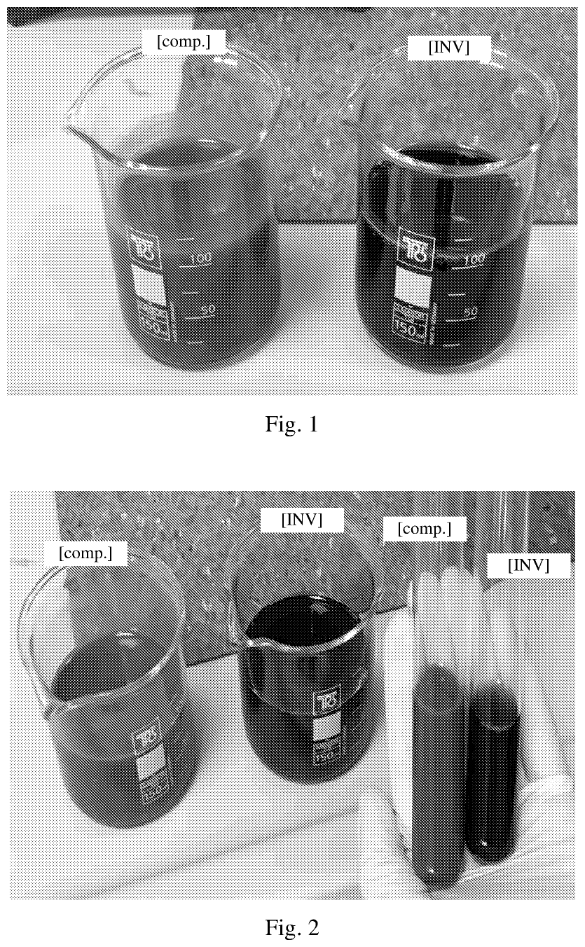

FIG. 1 and FIG. 2 show the two compositions compared.

In particular, FIG. 1 shows the two compositions just prepared in the respective beckers.

Part of each composition was then poured into a test tube (FIG. 2, right) in order to show with further clarity that the composition [comp.] has a cloudy and uneven appearance, while the composition [INV] has a clear and homogeneous appearance.

On the left of FIG. 2, the respective starting beckers can be seen, photographed about 15 minutes after FIG. 1. It can be observed that the composition [comp.] already had the first whitish sediments on the bottom, a sign that this composition is not stable even after a few minutes. Differently, the composition [INV] is unaffected.

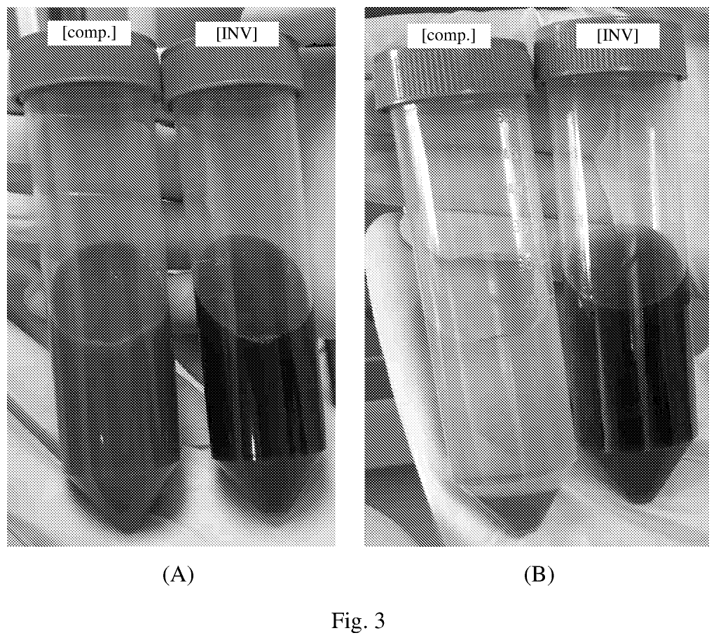

The compositions were also subjected to a centrifugation test at 3000 rpm for 5 minutes. FIG. 3A shows the tubes before centrifugation, while FIG. 3B shows the tubes after centrifugation. It can easily be observed that in the composition [comp.] there was a quantitative separation of the components with sediment formation, while the composition [INV] remained perfectly clear and homogeneous.

Therefore, the crucial importance is evident not only of the association of components as claimed, but also of the weight ratio between plant extracts and Lactobacilli, since above 10:1, the composition drastically reduces its stability.

1. Composition comprising:
at least one Lactobacillus selected from Lactobacillus rhamnosus, Lactobacillus reuteri and mixtures thereof,
at least one plant extract selected from tamarind extract, sugar cane extract and mixtures thereof, wherein said at least one plant extract and said at least one Lactobacillus are in a weight ratio of 1.1:1 to 10:1.

2. The composition of claim 1, wherein said at least one plant extract and said at least one Lactobacillus are in a weight ratio of 1.2:1 to 5:1.

3. The composition of claim 1, comprising up to 5 wt % of said at least one Lactobacillus, based on the weight of the composition, preferably 0.1-3 wt %, said at least one Lactobacillus being in a concentration of 10-200 billion CFU/g.

4. The composition of claim 1, comprising up to 10 wt % of said at least one plant extract, based on the weight of the composition, preferably 0.2-6 wt %.

5. The composition of claim 1, comprising a mixture of Lactobacillus rhamnosus and Lactobacillus reuteri, wherein preferably Lactobacillus rhamnosus is in greater amount than Lactobacillus reuteri, more preferably, Lactobacillus rhamnosus and Lactobacillus reuteri are in weight ratio of 4:1 to 1.2:1.

6. The composition of claim 5, comprising up to 3 wt % of Lactobacillus rhamnosus, preferably 0.2-2 wt %, and comprising up to 2 wt % of Lactobacillus reuteri, preferably 0.1-1.5 wt %, based on the weight of the composition.

7. The composition of claim 1, wherein said at least one Lactobacillus is tyndallized.

8. The composition of claim 1, comprising a mixture of tamarind extract and sugar cane extract.

9. The composition of claim 8, comprising up to 8 wt % of tamarind extract.

10. The composition of claim 1, further comprising at least one alga extract.

11. The composition of claim 10, in the form of an aqueous spray, comprising:
0.2-2 wt % of tyndallized Lactobacillus rhamnosus
0.1-1.5 wt % of tyndallized Lactobacillus reuteri
0.5-6 wt % of sugar cane extract
0.1-1 wt % of tamarind extract
0.1-1 wt % of mixture of Laminaria digitata extract and Chlorella vulgaris extract.

12. The composition of claim 1, further comprising vitamin E, preferably up to 2 wt %, based on the weight of the composition.

13. The composition of claim 12, in the form of an oily suspension, comprising:
0.2-2 wt % of Lactobacillus rhamnosus
0.1-1.5 wt % of Lactobacillus reuteri
0.5-6 wt % of sugar cane extract
0.1-1 wt % of tamarind extract
up to 2 wt % of vitamin E.

14. Tyndallized Lactobacillus for use in the prevention and topical treatment of skin diseases, preferably in pet animals, said Lactobacillus being selected from Lactobacillus rhamnosus, Lactobacillus reuteri and mixtures thereof.

15. A method for the topical external treatment of skin diseases, the method comprising the step of administering via topical external route the composition of claim 1 to a subject in need thereof.
